(19) United States
(12) Patent Application Publication
(10) Pub. No.: US 2006/0036155 A1
(43) Pub. Date: Feb. 16, 2006

(54) Method for positioning a medical image slice

(76) Inventor: Stefan Assmann, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/199,934

(22) Filed: Aug. 9, 2005

(30) Foreign Application Priority Data
Aug. 9, 2004 (DE) 10 2004 038 616.1

(51) Int. Cl. A61B 5/05

(52) U.S. Cl. 600/410

(57) ABSTRACT

In a method for positioning an image slice of an examination subject obtained with a diagnostic imaging apparatus, the determination of the position ensues automatically according to settable criteria. The criteria are established using an already-acquired slice or slice series.

### BACKGROUND OF THE INVENTION

1. Field of the Invention

The present invention concerns a method for positioning an image slice of an examination in a medical diagnostic imaging apparatus.

2. Description of the Prior Art

By means of medical diagnostic imaging methods, it is possible to graphically show pathological defects in the human body. The images support a treating physician in making a medical finding. For this purpose, it is necessary to show the pathological defect (for example a defect in the region of the human heart) with optimal detail and optimal placement in one or more images.

In heart function examinations by means of magnetic resonance tomography, it is possible, for example, to automatically establish in which region of a human heart a pathological defect of the heart wall exists. For this purpose, the thickness of the heart wall is automatically-analyzed using a number of magnetic resonance images that show a temporal cycle (and therewith a movement) of the heart, and locations in the heart wall with lower thickness change from image to image are marked. This functionality is contained in the syngo Argus Viewer commercially available from Siemens. It is now desirable, for example, to implement a perfusion or vitality measurement at precisely the determined location in order to show with optimal detail the possibly present pathological defect. For this purpose, a slice of a subsequent magnetic resonance measurement must be placed at the thinnest point of the heart wall, which conventionally ensues manually. This procedure is intricate and leads to long examination times for the physician and patient.

### SUMMARY OF THE INVENTION

It is an object of the present invention to provide a method that makes such procedures easier.

The above object is achieved in accordance with the present invention by a method for positioning a slice of a subject, for acquiring data from the slice with a diagnostic imaging apparatus, including the steps of establishing a positioning criterion, analyzing a data set dependent on the positioning criterion, and determining a position for the slice that fulfills the positioning criterion.

The position determined according to settable criteria is thereby automatically determined according to known methods using an already-acquired slice or slice series and is transferred to the imaging diagnostic apparatus. A measurement (data acquisition) is then implemented at this position. The physician thereby saves the time otherwise necessary to manually position the slice.

In an embodiment of the method, a pathological defect is defined using the criteria and an existing pathological defect is automatically established in the already-acquired slice or slice series. The position of the slice to be measured is subsequently placed so that the pathological defect is contained in the slice. Measurement processes are simplified for the physician and automated as much as possible with this method.

In a further embodiment of the method, the determination of the position is implemented during the measurement (data acquisition) for a different slice. Additional time can thereby be saved. The position of the further slice is thus available to the treating physician immediately after the conclusion of the measurements of a slice, such that the measurement can begin following immediately afterwards. The examination time is thereby shortened.

### DESCRIPTION OF THE DRAWINGS

FIG. 1 is a schematic flowchart of an embodiment of the method in the example of a heart function examination.

FIG. 2 schematically illustrates a magnetic resonance image after determination of a heart defect.

### DESCRIPTION OF THE PREFERRED EMBODIMENTS

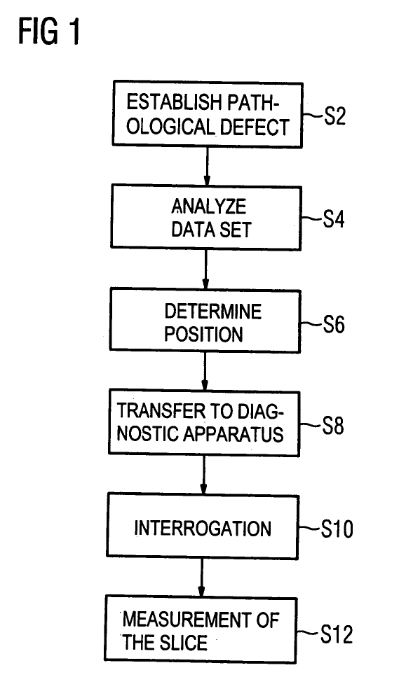

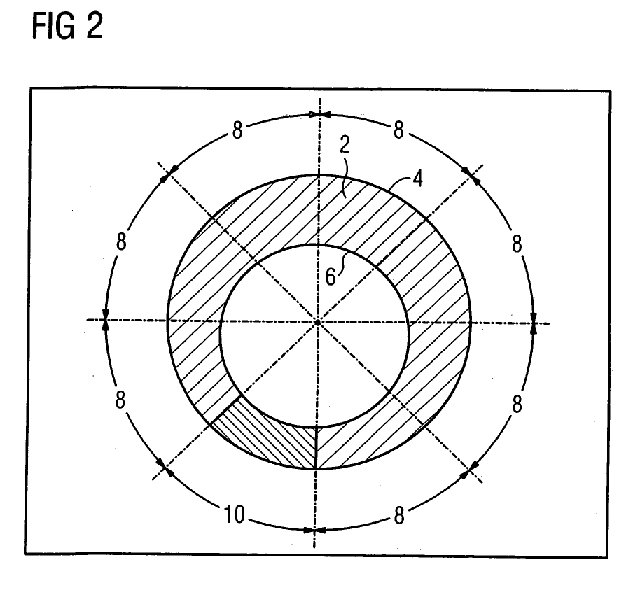

According to FIG. 1, in a first method step S2 a criterion is initially defined using which a heart defect of a patient to be examined is established. As is explained using FIG. 2, this can be a minimal thickness increase of the heart wall within a slice series, the slice series mapping a movement cycle of the heart. In a second method step S4, an already-acquired slice or slice series is thereupon examined for the presence of a pathological defect. For this purpose, the thickness of the heart wall is automatically analyzed and a possibly present point (location) with low thickness variation is determined. This is further explained below using FIG. 2.

In a third method step S6, the position of a slice or of a slice series is automatically determined that optimally maps the corresponding point (location) of the heart wall. For this purpose, the spatial coordinates at the point of the heart wall are determined. In a fourth method step S8, the coordinates are transferred to a magnetic resonance apparatus with which the subsequent measurements should be implemented. In a fifth method step S10, an interrogation occurs wherein the physician can manually change parameters of the measurement in the event that this appears necessary. The physician can thus change the number of the slices or the temporal sequence of the slices to be measured. It is likewise possible for the physician to plan the measurement. Thus, for example, perfusion or vitality measurements can be performed at the determined point of the heart wall in order to more precisely test the functionality. It is likewise possible to make high-resolution images of the determined point in order to, for example, determine the dimensions of the location. In a sixth method step S12, the slice is measured.

FIG. 2 shows a magnetic resonance image of a heart wall 2 whose thickness change has already been analyzed. For this purpose, within a slice series with slices of the same position acquired in temporal succession, the heart wall 2 is described by two closed curves 4 and 6 and the change of the separation of the two curves 4 and 6 within the slice series is automatically analyzed. To show the result, the heart wall 2 is sub-divided into segments 8 and 10 that exhibit different colors, which is not shown here. A blue-colored segment 8 thus indicates that the thickness change of the heart wall 2 is sufficiently large in this segment 8. In a red-colored segment 10, the thickness change is small, which indicates a pathological defect. Local variations of the thickness change are shown by a color gradient from red to blue so that the point with the smallest thickness change can be determined. Further examinations should occur at precisely this point, which is why the corresponding position of the slice is determined and transmitted to the magnetic resonance apparatus according to the method described in FIG. 1.

The described exemplary embodiment refers only to measurements with a magnetic resonance apparatus, but the method is likewise applicable for other imaging modalities, for example computed tomography.

With the described method, it is made possible for the physician to implement complex diagnostic methods composed of a number of steps with little effort, which methods make a generation of a more detailed medical finding easier. For the patient, the method likewise offers the advantage of a faster flow of the examination. Further measurements by the physician can thus be conducted during the analysis of the already-acquired slice or slice series, since the analysis ensues automatically.

Although modifications and changes may be suggested by those skilled in the art, it is the intention of the inventor to embody within the patent warranted hereon all changes and modifications as reasonably and properly come within the scope of his contribution to the art.

I claim as my invention:

1. A method for positioning a slice in a subject for data acquisition from the slice with a diagnostic imaging apparatus, comprising the steps of:
establishing a position criterion;
non-manually, electronically analyzing a data set dependent on said positioning criterion; and
non-manually, electronically determining a position in said data set that fulfills said positioning criterion.

2. A method as claimed in claim 1 comprising defining a pathological defect in the subject using said positioning criterion, automatically electronically establishing said pathological defect in said data set and automatically positioning said slice within said data set so that said pathological defect is contained in said slice.

3. A method as claimed in claim 1 comprising acquiring imaging data from said slice.

4. A method as claimed in claim 3 comprising acquiring said imaging data from said slice only upon manual authorization.

5. A method as claimed in claim 3 comprising allowing manual entry of data acquisition parameters into said diagnostic imaging apparatus for acquiring said data from said slice.

6. A method as claimed in claim 1 comprising employing a magnetic resonance apparatus as said diagnostic imaging apparatus.
